(19) United States
(12) Patent Application Publication
(10) Pub. No.: US 2015/0099971 A1
(43) Pub. Date: Apr. 9, 2015

(54) DEVICE FOR MEASURING AN ULTRASONIC OR BIOMECHANICAL PARAMETER OF A VISCOELASTIC MEDIUM

(71) Applicant: ECHOSENS, Paris (FR)

(72) Inventors: Laurent Sandrin, L'hay les Roses (FR); Véronique Miette, Villejuif (FR); Magali Sasso, Paris (FR); Jennifer Oudry, Malakoff (FR); Ludovic Fradin, Paris (FR)

(73) Assignee: ECHOSENS, Paris (FR)

(21) Appl. No.: 14/397,075

(22) PCT Filed: Apr. 26, 2013
(86) PCT No.: PCT/EP2013/058800

§ 371 Date: Oct. 24, 2014

(30) Foreign Application Priority Data
Apr. 27, 2012 (FR) 1253904

(51) Int. Cl. A61B 8/00; A61B 8/08

(52) U.S. Cl. CPC A61B 8/4236; A61B 8/0858; A61B 8/4483
USPC 600/438

(57) ABSTRACT

A device for measuring an ultrasonic or biomechanical parameter of a viscoelastic medium, includes an ultrasound transducer; at least one vibrator having a fixed part and a mobile part, the ultrasound transducer being secured to the mobile part of the at least one vibrator; at least one adhesive element secured to the vibrator, the adhesive element being configured to be fixed adhesively to a surface facing it belonging to the viscoelastic medium and to keep the emission and reception face of the ultrasound transducer facing the surface.

### TECHNICAL DOMAIN OF THE INVENTION

This invention relates to a device for measuring an ultrasonic or biomechanical parameter characteristic of a viscoelastic medium, such as the elasticity or viscosity of a soft human or animal tissue, or more generally measurement of any parameter of a viscoelastic medium with a backscattered ultrasound signal after ultrasonic illumination, such as signal attenuation. It is particularly but not exclusively applicable to measurement of the elasticity of adipose tissues of a human or an animal.

### STATE OF PRIOR ART

A method is known for observing propagation of a low frequency shear pulse wave simultaneously at a large number of points in a diffusing viscoelastic medium. This is done by emitting ultrasound compression waves at high velocity to obtain a sequence of measurements in the medium, and the measurements thus obtained are then processed off-line so as to determine movements of the medium during propagation of the shear wave.

Ultrasound transducers are used in existing devices to generate low frequency shear waves by vibrating mechanically. Patent application FR2843290 describes a device for measuring the elasticity of an organ that emits an ultrasound signal after illumination by ultrasound, the device comprising an ultrasound transducer and an electrodynamic actuator slaved so as to make the transducer vibrate at low frequency.

However, such a device has disadvantages. An operator is necessary to position and to hold the device such that the ultrasound transducer is facing the tissue and different inclinations of the ultrasound transducer relative to the organ give different measurement results. In other words, the correct position of the ultrasound transducer depends on the skill of the operator.

Furthermore, when the device pressed on soft tissues, the weight of the device modifies the tissue properties and distorts the results.

### GENERAL DESCRIPTION OF THE INVENTION

The purpose of the invention is to overcome the disadvantages of the previously described device according to prior art by providing a device for measuring an ultrasonic or biomechanical parameter of a viscoelastic medium that does not create any significant change to parameters of the viscoelastic medium and for which measurements do not depend on the operator's skills.

To achieve this, it discloses a device for measurement of an ultrasonic or biomechanical parameter of a viscoelastic medium, said device comprising at least one ultrasound transducer, said device being characterised in that it comprises:
at least one vibrator with a fixed part and a moving part, said ultrasound transducer being fixed to said moving part of said at least one vibrator;
at least one adhesive element fixed to the vibrator, said adhesive element being configured so that it is fixed by adhesion onto a surface facing the viscoelastic medium and holds the emission and reception face of the ultrasound transducer facing the surface.

Non-limitatively, a surface facing the viscoelastic medium means a surface separating the emission and reception face of the ultrasound transducer from the viscoelastic medium;
for example, when it is required to measure ultrasonic or biomechanical properties of adipose tissue (adipose tissue being the viscoelastic medium in this example), the epidermis is the surface facing the viscoelastic medium, or
for example, when it is required to measure the ultrasonic and/or biomechanical properties of the epidermis (the epidermis being the viscoelastic medium in this example), the external surface of the epidermis forms the surface facing the viscoelastic medium.

In these non-limitative examples, the surface facing the viscoelastic tissue is composed of a thin biological tissue.

An adhesive element non-limitatively refers to a patch, an adhesive tab, self-sticking tape or any other means comprising an adhesive face that can be fixed by sticking onto a biological tissue such as the epidermis or an organ.

Due to the adhesive element (indifferently called retaining means), an operator fixes the device onto the epidermis such that the ultrasound emission and reception face of the ultrasound transducer is facing the viscoelastic medium and there is no need to touch it throughout the duration of the measurements. The vibrator is chosen to be small and lightweight, for example such as a loudspeaker, a scale model electrodynamic actuator or a piezoelectric motor, so that the device can be easily handled and operated so as not to significantly modify the properties of the medium when the device is placed facing the medium.

The device according to the invention can advantageously be used to make an elastography measurement without handling the device, or even without the operator taking any action during the measurement. Thus, the results obtained do not depend on the skill of the operator. In other words, for the same medium, measurements obtained will be identical regardless of which operator did the work because the operator does not hold the device.

Furthermore, such a device can for example be directly in contact with an organ before a transplant, or an organ not protected by the ribs in order to determine the ultrasound attenuation or even spectral parameters as ultrasonic parameters.

Apart from the characteristics that have just been mentioned in the previous section, the device according to the invention can have one or several complementary characteristics among the following, taken individually or in any technically possible combination:
the at least one vibrator is a loudspeaker capable of vibrating;
the adhesive element is fixed onto the fixed part of the vibrator and it has a free adhesive end configured to be fixed by adhesion onto the surface facing the viscoelastic medium and hold the emission and reception face of the ultrasound transducer facing the surface;
the adhesive free end is located around and at the periphery of the vibrator;
in one embodiment:
   the vibrator comprises at least two support tabs, each of said two tabs comprising a first end fixed to the fixed part of the vibrator and a second end configured to press on the surface facing the viscoelastic medium,
   an adhesive element is fixed onto each of the at least two support tabs and it has a free end configured to be fixed by adhesion onto the surface facing the viscoelastic medium and to hold the emission and reception face of the ultrasound transducer facing the surface;
in one embodiment:
   the vibrator comprises at least two support tabs, each of said two support tabs comprising a first end fixed to the moving part of the vibrator and a second end fixed to an ultrasound transducer, and in that
   an adhesive element is fixed onto the fixed part of the vibrator and has a free adhesive end located at the periphery of the vibrator and configured so that it can be fixed by adhesion onto the surface facing the viscoelastic medium and hold the emission and reception face of the ultrasound transducers facing the surface.
In one embodiment:
   the vibrator comprises at least two support tabs, each of said two support tabs comprising a first end fixed to the fixed part of the vibrator and a second end fixed to an ultrasound transducer, and in that
   an adhesive element is fixed onto the fixed part of the vibrator and has a free adhesive end located at the periphery of the vibrator and configured so that it can be fixed by adhesion onto the surface facing the viscoelastic medium and hold the emission and reception face of the ultrasound transducers facing the surface.
said device comprises a membrane capable of allowing ultrasounds to pass through without changing them, said membrane being inserted between the emission and reception face of at least one ultrasound transducer and the surface facing the viscoelastic medium;
the device comprises bonding means between the adhesive element and the vibrator;
the device comprises several ultrasound transducers;
the transducers are arranged in the form of a strip;
the ultrasound transducers are arranged on a circle, with the vibrator at the centre of the circle;

the central vibration frequency of the vibrator (13) is between 20 and 1500 Hertz;
the central frequency of the ultrasound transducer is between 0.5 and 40 MegaHertz.

The invention and its different applications will be better understood after reading the following description with reference to the figures that accompany it.

### BRIEF DESCRIPTION OF THE FIGURES

The figures are only shown for guidance and are in no way limitative of the invention. The figures show:

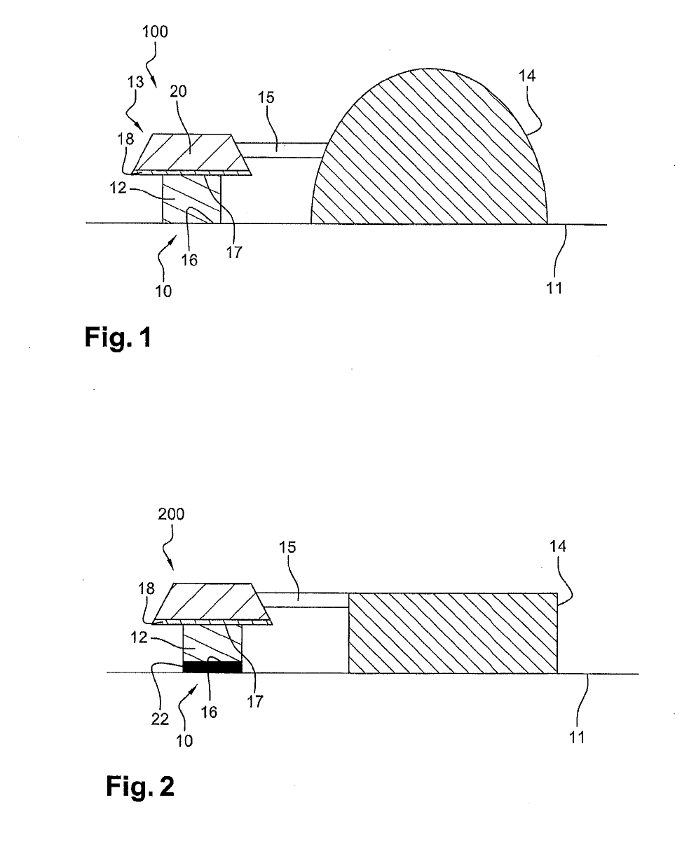

in FIG. 1, a diagrammatic view of a device according to a first embodiment of the invention;

in FIG. 2, a diagrammatic view of a device according to a second embodiment of the invention;

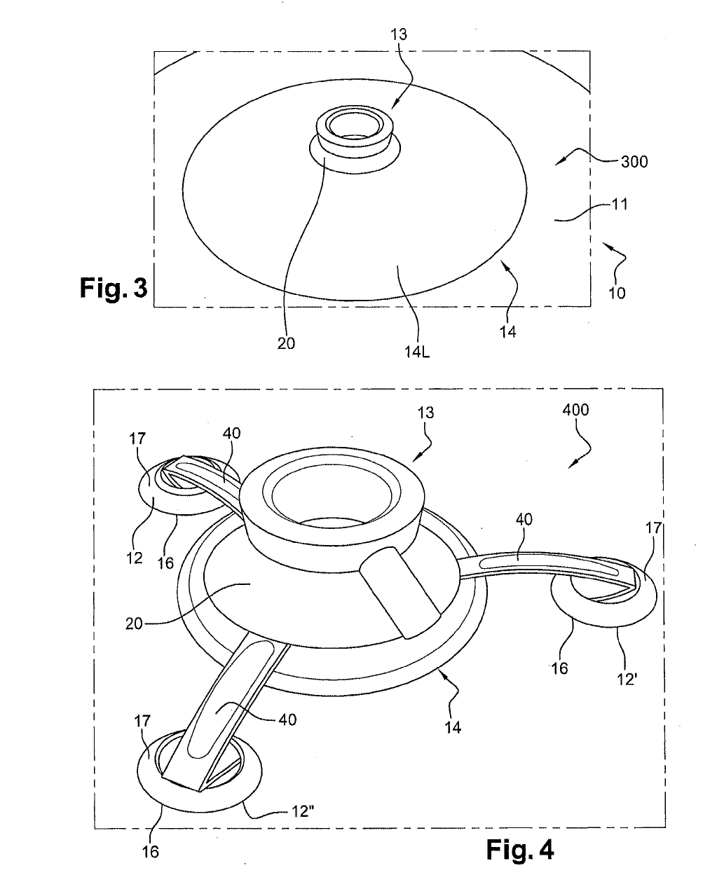

in FIG. 3, a diagrammatic view of a device according to a third embodiment of the invention;

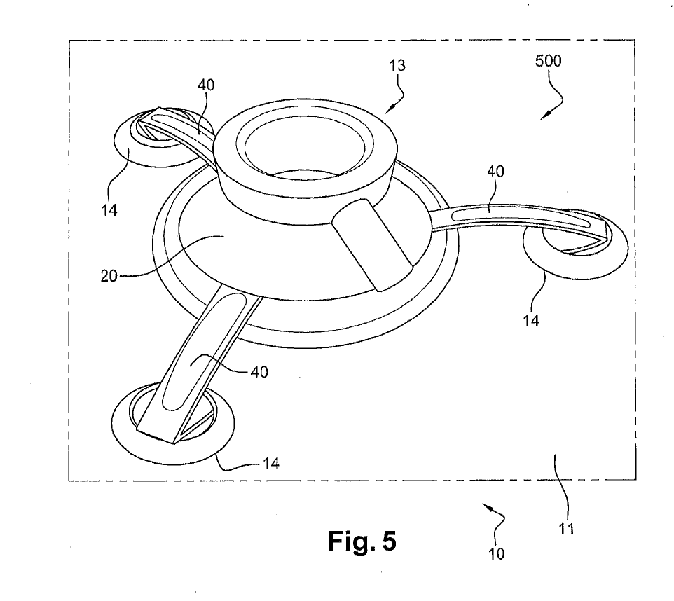

in FIG. 4, a diagrammatic view of a device according to a fourth embodiment of the invention;

in FIG. 5, a diagrammatic view of a device according to a fifth embodiment of the invention;

### DETAILED DESCRIPTION OF AT LEAST ONE EMBODIMENT OF THE INVENTION

Unless mentioned otherwise, any one element appearing on the different figures will have a single reference.

FIG. 1 is a diagrammatic view of the device 100 for measuring an ultrasound or biomechanical parameter of a viscoelastic medium 10 according to a first non-limitative embodiment of the invention, the parameter being correlated to a pathological condition of the medium. The device 100 comprises:
an ultrasound transducer 12 comprising a bottom face 16 formed by the emission and reception face of the ultrasound transducer 12 and a top face 17;
a vibrator 13 that in this non-limitative embodiment is a loudspeaker 13 comprising a fixed part 20 and a moving part 18, for example the vibrator 13 being capable of making transient or monochromatic type vibrations;
an adhesive element 14 fixed to the vibrator 13;
bonding means 15 between the adhesive element 14 and the vibrator 13 fixing the adhesive element 14 to the fixed part 20 of the vibrator 13.

It should be noted that in other embodiments, the vibrator 13 is a scale model electrodynamic actuator or a piezoelectric motor, in other words an inexpensive, compact and lightweight vibrator. Lightweight means a vibrator 13 for example with a weight of less than 150 grams and preferably less than 100 grams. In one interesting non-limitative embodiment, the weight of the vibrator is of the order of 30 grams.

The bottom face 16 (also called the emission and reception face 16) of the transducer is supported on a surface 11 (for example the epidermis) facing the viscoelastic medium 10 (for example the adipose tissue), and the moving part (for example formed by a membrane) 18 of the loudspeaker 13 is fixed to the top face 17 of the transducer 12. The adhesive element 14 bonds to the surface 11 (the epidermis in the example) facing the viscoelastic medium 10 (the adipose tissue in the example), and is fixed to the fixed part 20 of the loudspeaker 13 through the bonding means 15. Thus, the transducer and vibrator assembly is fixed to the epidermis. The bonding means 15 are composed for example of a rigid or flexible rod 15, one end of which is fixed to the adhesive element 14 and another end is fixed to the body 20 of the loudspeaker 13. Non-limitatively, the rod 15 extends along an axis approximately parallel to the surface 11 of the viscoelastic medium 10.

Due to the adhesive element 14 and bonding means 15, the loudspeaker 13/transducer 12 assembly is held in a stable position relative to the viscoelastic medium 10, such that the bottom face 16 of the transducer 12 is in contact with the surface 11 facing the viscoelastic medium 10. It is understood that an ultrasound transducer protection gel or membrane that allows ultrasounds to pass may be placed between the bottom face 16 of the transducer 12 and the surface 11.

The loudspeaker 13 is adjusted such that its moving part 18 vibrates at low frequency at a defined central frequency f1. f1 is advantageously chosen to be between 20 and 1500 Hertz and more particularly between 70 and 100 Hz. Being fixed to the transducer 12, the membrane entrains the transducer 12 in low frequency vibration, that itself generates a low frequency pulse received by the viscoelastic medium. A pulse (or vibration) received by the viscoelastic medium 10 causes the propagation of a low frequency shear wave that propagates in the viscoelastic medium 10. The displacement velocity of the shear wave depends on the elasticity and viscosity of the viscoelastic medium 10.

It should be noted that the loudspeaker 13 can generate a single low frequency pulse or a plurality of successive low frequency pulses.

Furthermore, the ultrasound transducer 12 is capable of generating ultrasound waves at high frequency f2. The central frequency f2 of the transducer 12 is advantageously between 0.5 and 40 MegaHertz, for example 3.5 MegaHertz. This central frequency f2 is chosen as a function of the required penetration depth of ultrasound waves in the viscoelastic medium 10; as the frequency increases, the distance by which waves penetrate into the viscoelastic medium 10 reduces. For example, at 12 MegaHertz, measurements are made on a depth of the order of 5 mm under the epidermis. Ultrasound waves are reflected by particles in the medium, and the return signal is received by the same transducer 12.

The moving part 18 of the loudspeaker 13 is fixed to the top face 17 of the transducer 12, for example by gluing. Advantageously, a glued attachment is simple and inexpensive.

The method used subsequently to measure the elasticity of the viscoelastic medium extracted from ultrasound signals or ultrasound images is well known to those skilled in the art, and for example is described in detail in patent FR2843290.

In a second embodiment of a device 200 according to the invention shown in FIG. 2, a membrane 22 is inserted between the surface 11 facing the viscoelastic medium 10 and the emission and reception face 16 of the transducer 12. The emission and reception face 16 of the transducer is supported on the membrane 22. The membrane 22 is composed of a material that allows ultrasounds to pass through it without modifying them. This membrane 22 makes it possible to reuse the device 200, all that has to be replaced between two uses is the membrane 22. This special feature makes it possible to respect hygiene conditions imposed in hospitals. It is easy and inexpensive to replace membrane 22 only.

FIG. 3 shows a diagrammatic view of a device 300 for measuring the elasticity of the viscoelastic medium 10 according to a third non-limitative embodiment of the invention. In particular, the device 300 comprises:
an ultrasound transducer 12 (not shown on the figure), comprising a bottom face 16 and a top face 17;
a loudspeaker 13;
an adhesive element 14 fixed to the fixed part 20 of the vibrator 13 and with an adhesive free end 14L configured to be fixed by adhesion onto the surface 11 facing the viscoelastic medium 10 and hold the emission and reception face of the ultrasound transducer (not shown) facing the surface 11, the free end 14L in this example being formed by the peripheral edge of the adhesive element 14.

It should be noted that in this non-limitative embodiment, the adhesive free end 14L is located on the periphery of the vibrator 13, in other words around the periphery of the vibrator 13. In the example shown, the fixed part 20 of the vibrator is circular and the adhesive element 14 is circular. Obviously, another form of adhesive element could be imagined, for example it could be rectangular, triangular, or other.

Due to the small size of the device 300 according to the invention, it is possible to position several devices 300 on the surface 11 facing the viscoelastic medium 10 to perform simultaneous measurements.

FIG. 4 is a diagrammatic view of a device 400 for measuring the elasticity of the viscoelastic medium 10 according to a fourth non-limitative embodiment of the invention. The device 400 comprises:
four ultrasound transducers 12, 12′, 12″ (+one transducer that is not shown on FIG. 4 because it is fixed to the membrane of the loudspeaker 13), each comprising a bottom face 16 and a top face 17;
a loudspeaker 13;
an adhesive element 14 placed around the periphery of the loudspeaker 13;
support tabs 40 between the loudspeaker 13 and the transducers 12, 12′ and 12″.

In this embodiment, the ultrasound transducer that is not shown is fixed to the membrane of the loudspeaker 13, therefore the membrane is capable of entraining the ultrasound transducer 12 in low frequency vibration, that itself generates a low frequency pulse received by the viscoelastic medium. A pulse (or vibration) received by the viscoelastic medium 10 generates propagation of a low frequency shear wave, which propagates in the viscoelastic medium 10. This ultrasound transducer that is not shown can emit and receive ultrasound signals so as to determine the displacement velocity of the shear wave propagating in the tissues.

It is understood that the device 400 may comprise more than four ultrasound transducers.

In one non-limitative embodiment, the support tabs 40 are formed from several rigid rods 40, in which one end of each rod 40 is fixed to the fixed part 20 of the vibrator 13, and another end is fixed to one of the transducers 12, 12′ and 12″. It should be noted that rods 40 may be flexible. In this embodiment, since the support tabs 40 are fixed to the fixed part of the vibrator 13, no vibration is applied to the ultrasound transducers 12, 12′ and 12″ and they can be used in echograph mode.

In one non-limitative embodiment not shown, the support tabs 40 are formed from several rigid rods, in which one end of each rod 40 is fixed to the moving part of the vibrator 13 and another end is fixed to one of the transducers 12, 12′ and 12″. In this embodiment, since the support tabs 40 are fixed to the moving part of the vibrator 13, a vibration movement is applied to the ultrasound transducers 12, 12′ and 12″ when the moving part of the vibrator 13 emits one or several low frequency pulses, exactly like for the vibrator that is directly fixed to the membrane of the vibrator 18.

Due to the adhesive element 14 and the support tabs 40, the loudspeaker 13/ultrasound transducers assembly is held in a stable position relative to the viscoelastic medium, such that the bottom faces of the ultrasound transducers are pressed in contact with the surface 11 and extend approximately parallel to the surface 11. In this stable position, ultrasonic parameters may be measured at different locations in the viscoelastic medium without the operator touching the device. Thus, the measurements obtained do not depend on the skill of the operator.

It should be noted that in this non-limitative embodiment, the peripheral ultrasound transducers are arranged in the form of a circle with the vibrator and an ultrasound transducer at the centre of the circle but the peripheral ultrasound transducers could also be arranged in the form of a strip.

The use of a plurality of ultrasound transducers makes a plurality of simultaneous measurements possible. Since these measurements are made under the same conditions, they can be compared.

FIG. 5 shows a diagrammatic view of a fifth embodiment of the invention. The device 500 comprises:
an ultrasound transducer 12 (not shown) in contact with the surface 11 facing the viscoelastic medium 10;
a loudspeaker 13 comprising three support tabs 40, each support tab 40 comprising a first end fixed to the fixed part 20 of the vibrator 13 and a second end configured to be pressed against the surface 11 facing the viscoelastic medium 10;
an adhesive element 14 is fixed to the second end of each of the support tabs 40, each of the adhesive elements having a free end configured to be fixed by adhesion onto the surface 11 facing the viscoelastic medium 10 and to hold the emission and reception face of the ultrasound transducer facing the surface 11.

In general, the adhesive elements (namely the retaining means) are adapted to hold the emission and reception face 16, 16′ and 16″ of the transducer(s) 12, 12′ and 12″ facing a surface 11 facing the viscoelastic medium 10. In other words, the adhesive elements can fix the transducer(s) 12, 12′ and 12″ and consequently the vibrator 13 that is fixed to the transducer(s) 12, 12′ and 12″, to the surface 11 (for example the epidermis) facing the viscoelastic medium 10 (for example the adipose tissue located under the epidermis) for which the viscoelastic properties are to be determined. It is thus certain that the measurements obtained using the device are not operator-dependent and the results obtained will be the same for all operators. The transducer is not tilted or even displaced during the measurement.

Furthermore, the device according to the invention respects hygiene standards because the adhesive elements can be replaced after each use (for example, when the adhesive element is a medical type self-sticking tape).

This type of adhesive element is obviously not painful for the patient.

It should also be noted that the surface of the stomach of morbidly obese patients has a variable shape and a variable consistency (firm skin and protruding belly or soft belly with rolls of fat). The device can easily be positioned on such non-plane surfaces.

Obviously, the described embodiments are only given as examples. Those skilled in the art will be capable of making different variations of the device for measuring the elasticity of a viscoelastic medium, particularly in terms of the layout, number and arrangement of the ultrasound transducer(s) and the shape of the adhesive elements between the cup and the loudspeaker.

1. Device for measuring an ultrasonic or biomechanical parameter of a viscoelastic medium, said device comprising:
at least one ultrasound transducers;
at least one vibrator with a fixed part and a moving part, said ultrasound transducer being fixed to said moving part of said at least one vibrator;
at least one adhesive element fixed to the vibrator, said adhesive element being configured so that it is fixed by adhesion onto a surface facing the viscoelastic medium and holds an emission and reception face of the ultrasound transducer facing the surface.

2. The device according to claim 1, wherein the at least one vibrator is a loudspeaker capable of vibrating.

3. The device according to claim 1, wherein the adhesive element is fixed onto the fixed part of the vibrator and has a free adhesive end configured to be fixed by adhesion onto the surface facing the viscoelastic medium and hold the emission and reception face of the ultrasound transducer facing the surface.

4. The device according to claim 3, wherein the adhesive free end is located on the periphery of the vibrator.

5. The device according to claim 1, wherein
the vibrator comprises at least two support tabs, each of said two support tabs comprising a first end fixed to the fixed part of the vibrator and a second end configured to press on the surface facing the viscoelastic medium, and wherein
an adhesive element is fixed onto each of the at least two support tabs, each adhesive element having a free end configured to be fixed by adhesion onto the surface facing the viscoelastic medium and to hold the emission and reception face of the ultrasound transducer facing the surface.

6. The device according to claim 1, wherein
the vibrator comprises at least two support tabs, each of said two support tabs comprising a first end fixed to the moving part of the vibrator and a second end fixed to an ultrasound transducer, and wherein
an adhesive element is fixed onto the fixed part of the vibrator and has a free adhesive end located at the periphery of the vibrator and configured so that it can be fixed by adhesion onto the surface facing the viscoelastic medium and hold the emission and reception face of the ultrasound transducers facing the surface.

7. The device according to claim 1, wherein
the vibrator comprises at least two support tabs, each of said two support tabs comprising a first end fixed to the fixed part of the vibrator and a second end fixed to an ultrasound transducer, and wherein
an adhesive element is fixed onto the fixed part of the vibrator and has a free adhesive end located at the periphery of the vibrator and configured so that it can be fixed by adhesion onto the surface facing the viscoelastic medium and hold the emission and reception face of the ultrasound transducers facing the surface.

8. The device according to claim 1, comprising a membrane capable of allowing ultrasounds to pass through without changing them, said membrane being inserted between the emission and reception face of at least one ultrasound transducer and the surface facing the viscoelastic medium.

9. The device according to claim 1, comprising a bonding component means between the adhesive element and the vibrator.

10. The device according to claim 1, comprising several ultrasound transducers.

11. The device according to claim 10, wherein the ultrasound transducers are arranged on a circle, with the vibrator at the centre of the circle.

12. The device according to claim 1, wherein a central vibration frequency of the vibrator is between 20 and 1500 Hertz.

13. The device according to claim 1, the wherein a central frequency of the ultrasound transducer is between 0.5 and 40 MegaHertz.

14. The device according to claim 9, wherein the bonding component comprises a rod having a first end fixed to the adhesive element and a second end fixed to the vibrator.

15. The device according to claim 14, wherein the rod is flexible.
